(19) United States
(12) Patent Application Publication
(10) Pub. No.: US 2006/0128615 A1
(43) Pub. Date: Jun. 15, 2006

(54) Ghrh analogues

(76) Inventor: Pierrette Gaudreau, Brossard (CA)

Correspondence Address: GOODWIN PROCTER LLP;PATENT ADMINISTRATOR, EXCHANGE PLACE, BOSTON, MA, 02109-2881, US

(21) Appl. No.: 10/527,598

(22) PCT Filed: Sep. 17, 2003
(86) PCT No.: PCT/CA03/01418

§ 371 Date: Nov. 3, 2005

Related U.S. Application Data
(60) Provisional application No. 60/411,340, filed on Sep. 18, 2002.

(51) Int. Cl. C07K 14/60

(52) U.S. Cl. 514/12; 530/324

(57) ABSTRACT

The present invention relates to growth hormone-releasing hormone (GHRH) analogues. More particularly, the invention relates to synthetic GHRH analogues of 29 amino acids or more, exhibiting concomitantly an increased resistance to proteolysis and high binding affinity to human GHRH receptor in in vitro studies, in comparison with human native GHRH (1-29)NH₂. The present invention also relates to a pharmaceutical composition comprising any one of said GHRH analogues and to the use of these analogues for specific stimulation of in vivo GH release as well as preparation of a drug in the treatment of GH deficiency-related conditions. The present invention also provides for a method for initiating GHRH-induced biological actions in a mammal.

### FIELD OF THE INVENTION

This invention relates to the field of growth hormone-releasing hormone (GHRH) analogues. More particularly, the invention relates to GHRH analogues of 29 amino acids or more, exhibiting an increased resistance to proteolysis and having a relatively high binding affinity to human GHRH receptor in in vitro studies, in comparison with human native GHRH (1-29)NH₂.

### BACKGROUND OF THE INVENTION

Growth hormone (GH) is a somatotropic anterior pituitary hormone responsible for regulating growth and exerting anabolic functions, such as stimulating protein synthesis and accretion, and lipolysis. Until the mid 1980's, the only source of human GH (hGH) was from pituitary glands collected post mortem. Today, hGH is available in large quantities through genetic engineering.

GH promotes growth in children and plays an important role in adult metabolism. GH deficiencies in children are associated with growth retardation or failure while GH excess causes gigantism or acromegaly, respectively.

GH is produced in somatotroph cells of the anterior pituitary gland of mammals and secreted throughout life. It is mainly controlled in the brain by two hypothalamic peptides: GHRH, which stimulates its secretion and synthesis; and somatostatin, which inhibits them. A number of peripheral factors regulate GH secretion. Among them, insulin-like growth factor-1 (IGF-1) represents an important one as it is produced by the liver in response to GH and acts on the hypothalamus to exert a negative feedback on GH secretion.

Pharmaceutical agents that target the GH axis include synthetic GHRH that stimulates GH release; a somatostatin analogue, octreotide that inhibits GH release; recombinant human GH (somatotropin, somatrem) that is used to replace GH in a state of deficiency; and recombinant IGF-1 that is used to treat GH insensitivity (Laron-type dwarfism).

GH declines with age in every animal species that have been tested to date. In humans, the amount of GH after the age of 21 to 31 falls by about 14% per decade, so that the total 24-hour GH production rate is reduced in half by the age of 60. Humans thus daily produce GH at about 500 μg at 20 years of age, 200 μg at 40 years, and 25 μg at 80 years old.

With the availability of biosynthetic GH for prescription use in the US since 1985, GH replacement therapy has been the treatment of choice in cases of growth hormone deficiency. In the US, the number of children eligible for GH treatment ranges from 11,000, if strict criteria for GH deficiency are applied, to 1.3 million, if all those with heights below the third percentile are candidates. The respective cost of GH therapy would jump from $155 million to $20 billion per year if the less stringent criterion became the standard of care (Cuttler L. et al., 1996). So far, pediatricians in the US have shown gratifying restraint in prescribing GH for non-approved indications, since only 20,000 children are receiving GH therapy (Finkelstein, B. S. et al., 1998).

Another problem is the low patient compliance, as conventional biosynthetic GH has to be injected. The complex amino acid structure of GH (191 amino acids) is completely destroyed in the gastrointestinal tract.

Overall, GH is contraindicated in patients with active malignant disease, benign intracranial hypertension, and proliferative or preproliferative diabetic retinopathy.

Growth hormone releasing hormone (GHRH) is a peptide of 44 amino acids. Several authors have reported that GHRH(1-29) NH₂, the 29 amino acid N-terminus fragment of GHRH(1-44) NH₂, exhibits the full bioactivity of GHRH(1-44) NH₂.

GHRH was first isolated from pancreatic tumours and subsequently from the hypothalamus of various mammals. In addition to the arcuate nucleus of the hypothalamus, GHRH is present in other hypothalamic nuclei such as the suprachiasmatic nucleus and in the other regions of the brain such as the limbic system. GHRH-like immunoreactivity and/or GHRH messenger ribonucleic acid (mRNA) has also been found in the placenta, gastrointestinal tract, ovary, testis, thymus, spleen and renal medulla.

GHRH binding sites have been localized and characterized in various tissue preparations and cell cultures from normal and tumoral pituitary, and from normal hypothalamus, testis, ovary and renal medulla. Pharmacological studies have demonstrated the existence of two populations of GHRH binding sites in the pituitary and ovary: a high affinity and low capacity binding site, corresponding to the physiologically relevant form of the receptor, and low affinity and high capacity binding site.

Alterations of the rat pituitary GHRH binding site parameters occur in the course of aging, leading to a loss of the high affinity binding sites.

GHRH is known to degrade rapidly in vivo. Degradation patterns of GHRH have been elucidated in serum and plasma, liver and target tissues such as the pituitary gland and hypothalamus. The vulnerable peptides identified so far are R2-R3, R10-R11, R11-R12, R14-R15, R18-R19, R20-R21, R21-R22 (BoulangeretaL Brain Res 1993; Boulanger et al. Peptides 1992). Furthermore, it is also known that modifications at these amino acid residues can prevent or decrease proteolysis as well as result in a longer duration of action of GHRH and its analogues (Girard P. et al. Eur J Clin Pharmacol 1987, 32: 507-513).

These caveats and limitations in naturally occurring GHRH resulted in the discovery of a new class of fourteen (14) polysubstituted synthetic GHRH superagonists, exhibiting a 5 to 13-fold increase in affinity to rat pituitary GHRH receptor, as described in U.S. Pat. No. 5,854,216. Such an invention provided non-toxic highly sensitive and selective marker peptides and marker polyclonal antibodies of the GHRH receptors.

In addition, GHRH analogues designed so far, either from academic organisations or pharmaceutical/biotechnology companies, were based on structural changes of these analogues aimed at merely improving their half-life in bioassays or in vivo experiments on animals.

To date, there is a need for GHRH analogues which, by simple amino acid polysubstitutions, can be modified to increase both their affinity to the pituitary GHRH receptor and their in vivo half-life. Furthermore, it needs to be demonstrated in vivo that the GHRH analogues will be able to stimulate GH secretion in animals and that they will be more potent than the native GHRH (1-44)-NH₂. In this connection, unexpected advantages were observed upon selection among the GHRH analogues described in U.S. Pat. No. 5,584,216.

### SUMMARY OF THE INVENTION

An object of the present invention is to provide GHRH analogues, which satisfy the above-mentioned need. Accordingly, the present invention relates to GHRH analogues, their use and a method for initiating GHRH-induced biological actions.

According to a first aspect, the invention is directed to a GHRH analogue, a derivative of said analogue, or a pharmaceutically acceptable salt thereof comprising formula X: Tyr-A2-Asp-Ala-Ile-Phe-Thr-A8-A9-A10-Arg-Lys-Val-Leu-A15-Gln-Leu-Ser-Ala-Arg-A21-A22-Leu-Gin-Asp-Ile-Met-Ser-Arg-A30-NH₂, wherein

A2 is Ala or D-Ala;

A8 is Asn, D-Asn or Ala;

A9 is Ser or Ala;

A10 is Tyr or D-Tyr;

A15 is Gly, Ala or D-Ala;

A21 is Lys or D-Lys;

A22 is Leu, D-Leu, Lys or Ala; and

A30 is a bond or any amino acid sequence of 1 up to 15 residues;

said analogue, derivative of said analogue or salt thereof having an in vitro potency index substantially higher than the in vitro potency index of a naturally occurring GHRH.

In another aspect, the invention is directed to a pharmaceutical composition comprising the above-mentioned analogue, derivative or salt thereof, and a pharmaceutically acceptable carrier.

In a further aspect, the invention is directed to the use of said analogues for the specific stimulation of in vivo release of GH.

In yet a further aspect, the invention is directed to the use of said analogues for the preparation of a drug in the treatment of GH deficiency-related conditions.

In yet another aspect, the invention is directed to a method for initiating GHRH-induced biological actions.

The invention and its advantages will be better understood upon reading the following non-restricted description of preferred embodiments thereof, made with references to the accompanying drawings.

### DESCRIPTION OF THE DRAWINGS

FIG. 1 shows a graphic representation of the secretion profile of rat growth hormone following a single intravenous injection of a GHRH analogue according to a preferred embodiment of the invention, at escalating doses versus natural human GRF(1-44)NH₂ peptide.

FIG. 2 shows a graphic representation of the secretion profile of rat growth hormone following a single subcutaneous injection of a GHRH analogue according to a preferred embodiment of the invention, at escalating doses.

FIG. 3 shows a graphic representation of the secretion profile of canine growth hormone following multiple subcutaneous injections of a GHRH analogue according to a preferred embodiment of the invention, at escalating doses.

### DESCRIPTION OF PREFERRED EMBODIMENTS

The originality of the present invention is directed to GHRH analogues that exhibit increased resistance to proteolysis and have a relatively high binding affinity to human GHRH receptor in in vitro studies, in comparison with human native GHRH (1-29)NH₂. The inventor has identified a general amino acid sequence of such a GHRH analogue. It will be understood that the term “GHRH analogue” means a GHRH agonist, more specifically a synthetic peptide that binds with high affinity to the GHRH receptor and increases plasma growth hormone (GH) concentration by stimulating somatotroph cells of the anterior pituitary gland to release GH.

The present invention also concerns compositions that comprise a GHRH analogue as defined herein and methods of use of such GHRH analogues and/or compositions.

GHRH Analogue, Derivative or Salt Thereof

According to the first aspect, the present invention relates to a GHRH analogue, a functional derivative or a pharmaceutically acceptable salt thereof. More specifically, the GHRH analogue of the invention has an amino acid sequence comprising the following Formula X: Tyr-A2-Asp-Ala-Ile-Phe-Thr-A8-A9-A10-Arg-Lys-Val-Leu-A15-Gln-Leu-Ser-Ala-Arg-A21-A22-Leu-Gln-Asp-Ile-Met-Ser-Arg-A30-NH₂, and wherein A2 is Ala or D-Ala; A8 is Asn, D-Asn or Ala; A9 is Ser or Ala; A10 is Tyr or D-Tyr; A15 is Gly, Ala or D-Ala; A21 is Lys or D-Lys; and A22 is Leu, D-Leu, Lys or Ala, and A30 is a bond or any amino acid sequence of 1 up to 15 residues. The term “residue”, when used with reference to an amino acid, means a radical derived from the corresponding aminoacid by eliminating the hydroxyl of the carboxyl group and one hydrogen of the amino group.

Furthermore, the GHRH analogue of the invention has an in vitro potency index substantially higher than the in vitro potency index of a naturally occurring GHRH. It will be understood that the expression “naturally occurring GHRH” encompasses both hGHRH (1-29)NH₂ (the functional portion of the native GHRH peptide) and hGHRH (1-44)NH₂ (the complete native GHRH peptide).

As used herein, the expression “in vitro potency index” represents a tool of comparison which results from multiplying i—the relative binding affinity of GHRH analogues compared with the native hGHRH (1-29)NH₂, in BHK cells expressing the hGHRH receptor; with ii—the relative resistance to in vitro proteolysis of compounds in comparison with hGHRH (1-29)NH₂ after preferably 60 or 180 minute-incubations in human plasma or human serum.

As used herein, the term “a relatively high binding affinity” means that the GHRH analogue of the invention has a binding affinity to human GHRH receptor of at least about 100-fold higher than the binding affinity of the native GHRH.

As used herein, the term “increased resistance to proteolysis” means that the GHRH analogue of the invention, upon in vitro incubation in human plasma or serum, has a substantially higher mean residual amount percentage, such as at least about 50%, in comparison with the native GHRH.

According to a preferred embodiment of the present invention, the expression “substantially higher”, used to characterize the in vitro potency index of the present GHRH analogue, derivative or salt thereof, indicates an in vitro potency index preferably at least 500-fold higher, more preferably 1500-fold higher and even more preferably 2500-fold higher than the in vitro potency index of the native hGHRH (1-29)NH₂.

As used herein the term “functional derivative”, as is generally understood, refers to a protein/peptide sequence that possesses a functional biological activity that is substantially similar to the biological activity of the GHRH analogue of the present invention. A functional derivative of a GHRH analogue of the present invention may or may not contain post-translational modifications such as covalently linked carbohydrate, if such modification is not necessary for the performance of a specific function. The term “functional derivative” encompasses the “fragments”, “segments”, “variants”, or “chemical derivatives” of a GHRH analogue as contemplated by the present invention.

As can be appreciated, Formula X is an amino acid (A) sequence. In general, the abbreviations used herein for designating the amino acids are based on recommendations of the IUPAC-IUB Commission on Biochemical Nomenclature (Biochemistry, 1972, 11: 1726-1732). More specifically, the term “amino acid” is described in general text books of peptide chemistry (Kipple, K. D, “Peptides and Amino Acids”, W. A. Benjamin, Inc., New York, 1966; “The Peptides”, E. D. Gross E. and Meienhofer J., vol. 1, Academic press, New York, 1979), and includes alanine, arginine, asparagine, aspartic acid, cysteine, glutamic acid, glutamine, glycine, histidine, hydroxylysine, hydroxyproline, isoleucine, leucine, lysine, methionine, phenylalanine, proline, pyroglutamic acid, sarcosine, serine, threonine, tryptophan, tyrosine and valine.

The GHRH peptides of the invention described herein have been synthesized preferably by using solid-phase peptide chemistry t-Boc-Acid-Labile protection scheme as described by Atherton E. L. Sheppard R. C. (“Solid-phase peptide synthesis: a practical approach”, IRL press, Oxford University press, Oxford, England, 1989, pages 1-203). It will be understood that GHRH analogues of the invention may be provided by any other methods known to one skilled in the art.

According to the present invention, different combinations of polysubstitutions in the native form of GHRH are preferred. Accordingly, in one such combination, a preferred GHRH analogue comprises the above-mentioned Formula X with the following substitutions: A2 is D-Ala, A8 is Ala, A15 is Ala, A22 is Lys. A9, A10, A21 and A30 are as defined hereinabove.

Another preferred analogue of the present invention comprises Formula X wherein A2 is D-Ala, A10 is D-Tyr, and A22 is Lys. A8, A9, A15, A21 and A30 are as defined hereinabove.

According to yet another preferred analogue of the present invention, said analogue comprises Formula X wherein A2 is D-Ala, A10 is D-Tyr, A15 is D-Ala and A22 is Lys. A8, A9, A21 and A30 are as defined hereinabove.

Pharmaceutical Composition

According to another aspect, the present invention relates to a pharmaceutical composition comprising a pharmaceutically effective amount of a GHRH analogue, functional derivative or salt thereof as described hereinabove, and a pharmaceutically acceptable carrier.

The term “composition” as used herein is intended to encompass a product comprising the GHRH analogue of the invention in the desired amounts. By “pharmaceutically acceptable”, it is meant that the carrier, diluent or excipient must be compatible with the GHRH analogue of the formulation and can be administered into a host without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, lactose, colors and the like. A preferable pharmaceutically acceptable carrier contemplated by the present invention is a saline solution, such as sodium chloride, preferably used at 0.9% or lactose used for the preparation of dry powder formulations intended for inhalation.

Methods of Use

According to other aspects of the present invention, the present invention relates to the use of the GHRH analogue of the invention or a pharmaceutical composition comprising same for the specific stimulation of in vivo release of GH, as well as for the preparation of a drug in the treatment of GH deficiency-related conditions. By “treatment”, it is meant both therapeutic treatment and prophylactic or preventative measures. Those in need of treatment include those already with the disorder or GH deficiency as well as those prone to have the disorder or GH deficiency, or those in which the disorder or GH deficiency is to be prevented.

According to the present invention, the expression “specific stimulation of in vivo release of GH” refers to the action of a GHRH analogue of the invention which activates GH release by direct binding to the GHRH receptor, but which does not activate GH release by direct binding to other receptor molecules, in a sample containing a mixed population of receptors.

GH deficiency-related conditions of the present invention encompass but are not limited to the following: hypothalamic pituitary dwarfism, burns, osteoporosis, renal failure, non-union bone-fracture, acute/chronic debilitating illness or infection, wound healing, post-surgical problems, lactation failure, infertility in women, cachexia in cancer patients, anabolic and/or catabolic problems, T-cell immunodeficiencies, neurodegenerative conditions, GHRH receptor-dependent tumors, aging, sleep disorders, muscle wasting diseases. As used herein, muscle wasting diseases could be any one of the following: sarcopenia, frailty in the elderlies, HIV and cancer. More specifically, use of the present pharmaceutical composition could be aimed at cancer patients who present side effects related to chemotherapy and radiotherapy.

In yet another aspect, the present invention provides a method for initiating GHRH-induced biological actions in a mammal. The method comprises the step of administering, to the mammal, an effective amount of a GHRH analogue, a functional derivative of said analogue or a pharmaceutically acceptable salt thereof, as defined herein, or of a pharmaceutical composition as defined above.

The expression “GHRH-induced biological actions” as used herein encompasses but is not limited to the following: regulation of sleep, regulation of food-intake and increase in protein synthesis. The increase in protein synthesis observed in the present invention, following GHRH analogue administration, could translate into an increase in muscle mass or an increase in milk production, among others, as described in Lapierre H. et al. (1995). J. Dairy Sci. 78: 804-815; Dubreuil, P. et al. (1996) Can J. Vet. Res. 60(1): 7-13; Lapierre H. et al. (1992) J. Anim. Sci. 70(3): 764-772; and Farmer C. et al. (1992) Biol. Neonate 61(2): 110-117.

As used herein the term “mammal” refers to any animal classified as a mammal, including humans, domestic and farm animals, and zoo, sports, or pet animals, such as dogs, horses, cats, cows, pigs, etc, in whom modulation of GHRH receptor activity is desired. “Modulation”, as used herein, is intended to encompass agonism, and/or partial agonism.

The term “effective amount” means the amount of GHRH analogue that will elicit the biological or clinical response of a tissue, system, animal or human that is being sought by the researcher, veterinarian, medical doctor or other clinician. In other words, such an effective amount of a compound for treating a particular disease is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. The terms “administration of a” and “administering a” compound should be understood to mean providing a GHRH analogue of the invention or a composition of the invention to the individual in need of treatment.

The GHRH analogue and the composition of the invention may be given to a mammal through various routes of administration. For instance, the composition may be administered in the form of sterile injectable preparations, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, or by intramuscular injection or by infusion. The GHRH analogue and the composition of the invention may also be formulated as creams, ointments, lotions, gels, drops, suppositories, sprays, liquids or powders for topical administration. They may also be administered into the airways of a subject by way of a pressurized aerosol dispenser, a nasal sprayer, a nebulizer, a metered dose inhaler, a dry powder inhaler, or a capsule. Suitable dosages will vary, depending upon factors such as the amount of each of the components in the composition, the desired effect (fast or long term), the disease or disorder to be treated, the route of administration, the bioavailability, and the age and weight of the mammal to be treated. In any event, for administering the GHRH analogue and the composition of the invention, methods well known in the art may be used.

### EXAMPLES

The following examples illustrate the wide range of potential applications of the present invention and are not intended to limit its scope. Modifications and variations can be made therein without departing from the spirit and scope of the invention. Although any methods and materials similar or equivalent to those described herein can be used in the practice for testing the present invention, the preferred methods and materials are described.

### Example 1

### Initial Selection of GHRH Analogues Based Upon In Vitro Data from GHRH Receptor Binding Affinity

Initial selection of a candidate from the original 14 polysubstituted GHRH analogues described in the U.S. Pat. No. 5,854,216 was based upon in vitro data on receptor affinity in 2-month old male Sprague Dawley rat anterior pituitary preparations. The new invention is based on the affinity of selected GHRH analogues for the human GHRH receptor (hGHRH-R) in baby hamster kidney (BHK) cells transfected with hGHRH-R, and on resistance to proteolysis in rat serum, human plasma or human serum. More precisely, the preferred drug candidates were selected, as compared hGHRH(1-29)-NH₂, for: i—their increased relative binding affinity to hGHRH(1-44)-NH₂ binding sites in rat anterior pituitary in vitro as well as to hGHRH-R in BHK-expressing cells in vitro; and ii—their relative resistance to proteolysis in vitro.

As can be noted from Table 1 below, the relative binding affinity of the synthetic peptides with the rat GHRH receptor is not predictive of the relative binding affinity with the human receptor. As will be noted, from this point forward, GHRH analogues as presented in Table 1 will be referred to as GHRH analogues # 1 to 5.

TABLE 1
Priority selection based on the expected theoretical combined effects of receptor
affinity and in vitro resistance to proteolysis on the overall bioactivity of
GHRH analogues in rat anterior pituitary membrane preparations and rat serum,
respectively, and of receptor affinity in BHK cell membrane preparations.

| | | | Relative binding | |
|---|---|---|---|---|
| | | | affinity in | Relative |
| | | Relative binding | hGHRH-R BHK- | resistance |
| | | affinity in rat | expressing | to proteolysis |
| No. | Structure | anterior pituitary*† | cells*† | in vitro |
| 1 | [D-Ala², Ala⁸, Ala¹⁵, Lys²²] | 13.33 ± 0.31 | 499 ± 234 | 1.87 |
| | hGHRH(1-29)-NH₂ | | | |
| 2 | [Ala⁸, Ala⁹, Ala¹⁵, Ala²²] | 7.74 ± 3.49 | 3.70 ± 0.52 | 1.81 |
| | hGHRH(1-29)-NH₂ | | | |
| 3 | [D-Ala², D-Tyr¹⁰, Lys²²] | 4.90 ± 2.70 | 239 ± 55 | 2.25 |
| | hGHRH(1-29)-NH₂ | | | |
| 4 | [D-Ala², Ala⁸, D-Tyr¹⁰, Ala¹⁵, D-Lys²¹, | 5.00 ± 0.91 | 0.05 ± 0.01 | 6.06 |
| | Lys²²] hGHRH(1-29)-NH₂ | | | |
| 5 | [D-Ala², D-Tyr¹⁰, D-Ala¹⁵, Lys²²] | 1.04 ± 0.40 | 939 ± 249 | 3.13 |
| | hGHRH(1-29)-NH₂ | | | |

GHRH analogue numbers in Table 1 correspond to numbers 13, 11, 7, 14 and 8 in Table 11 on pages 27-28 of the U.S. Pat. No. 5,854,216, respectively. *, values compared to hGHRH(1-29)-NH₂;†, use of [¹²⁵I-Tyr¹⁰] hGHRH(1-44)-NH₂ as a radioligand in structure-affinity studies.

### Example 2

### Processing of the Native GHRH and GHRH Analogues of the Present Invention—Experimental Assays

1—Competitive Binding Assay

¹²⁵I-GHRH binding assay was performed as previously described (Boulanger L, et al. (1999) Neuroendocrinology 70: 117-127), using [¹²⁵I-Tyr¹⁰]hGHRH(1-44)NH₂ as radioligand. Competition experiments were done in BHK (baby hamster kidney) 570 cell membrane preparations (25 μg of protein/assay tube) with increasing concentrations (0-1000 nM) of human(h)GHRH(1-29)NH₂, hGHRH(1-44)NH₂ or GHRH analogues, in a total volume of 300 μl 50 mM Tris-acetate buffer (pH 7.4), containing 5 mM MgCl₂, 5 mM EDTA and 0.42% BSA. Non specific binding was determined in presence of 1 μM hGHRH(1-29)NH₂. Incubation was carried out at equilibrium (23° C., 60 min) and stopped by centrifugation (12,000 g, 5 min, at 4° C.). The radioactivity content in pellets was determined by gamma counting. The affinity of hGHRH(1-29) NH₂ was tested in each experiment to assess the validity of the assay and determine the relative affinity of the analogues. The Ligand computerized program was used to analyze competition curves of GHRH analogues reported in Tables 2 and 3 and to determine their IC₅₀ (Gaudreau P. et al. (1992) J Med Chem, 35: 1864-1869).

2—In Vitro Proteolysis Assay in Serum and in Plasma

Ten μl of a 300 μM solution of hGHRH (1-29)NH₂ or of a GHRH analogue was solubilized in dimethysulfoxide (DMSO) and incubated in one of the following conditions: a—190 μl serum (1/100 dilution in picopure water) from 2-month-old male Sprague Dawley rats, at 37° C. for 0, 8, 15, 30 or 60 min, in polypropylene tubes; b—190 μl of human healthy volunteer plasma (from Human Whole Blood Na EDTA, males, drug free (Algorithme Pharma Inc.); project: MTL-P2-155; Lot: MTLP2155-01, supplied by LAB Dev Int); and c—190 μl of human healthy volunteer pooled serum, Lot: X409 (supplied by LAB Dev Int), at 37° C. for 0, 60, 120, 180 or 420 min, in polypropylene tubes. Proteolysis was stopped by adding 800 μl of ice-cold stop buffer (potassium-phosphate buffer, acidified to pH 0.8 with trifluoroacetic acid (TFA) and boiling 5 min (rat serum only). After centrifugation (12000 g, 5 min, 4° C.) (rat serum only), serum-peptide mixtures were passed through a conditioned Sep-Pak C-18 cartridge to extract native GHRH or a GHRH analogue residual concentrations from serum proteins. The native GHRH or the analogue was eluted in 2 ml of 50% acetonitrile-0.01% TFA/50% 0.01% aqueous TFA. Two hundred μl of extracted peptide, representing 1 μg of GHRH or analogue at time 0, was quantified by analytical HPLC, using one μ-Bondapak C18 column (10 μm particle size, 0.39×15 cm)(rat serum) or two C18 column in series (human serum and plasma) and a binary solvent system composed of NaClO4 0.01 M, pH 2.5 and acetonitrile. A linear gradient from 30 to 60% acetonitrile over 45 min (rat serum) or 30 to 50% (human serum and plasma) was used. Elution of intact peptide was monitored at 214 nm and residual concentration determined by assessment of peak surface areas (Boulanger L, et al. (1993) Brain Res 616: 39-47; Boulanger L, et al. (1992) Peptides 13: 681-68.9).

3—In Vivo Administration of Native GHRH or GHRH Analogue

The ability of human GHRH analogue # 5 (human [D-Ala², D-Tyr¹⁰, D-Ala¹⁵, Lys²²] GHRH (1-29)NH₂ analogue) to stimulate GH secretion was studied in adult female rats (26-34 weeks at onset of treatment) and in a male Beagle dog.

i—In Vivo Administration into Rats

Human GHRH analogue # 5 in 0.9% sodium chloride for injection USP was administered once either by intravenous (IV) or subcutaneous (SC) injection to female rats followed by a 14-day observation period, as shown in Table 2. Prior to administration, all dosing formulations were filtered using a 0.22 μm filter to ensure sterility. The actual amount of GHRH analogue # 5 administered was calculated and adjusted based on the animal's most recent body weight. Dosing started at approximately the same time each day, commencing at 9:00 am±30 minutes.

TABLE 2
In vivo administration of GHRH analogue # 5 to female rats.

| | | | Route | Number |
|---|---|---|---|---|
| Treatment | Dose Level | Dose Conc. | of Admin- | of |
| Group | (mg/kg) | (mg/ml) | istration | Animals |
| 1 (Negative | 0 | 0 | SC | 4 |
| Control *) | | | | |
| 2 | 0.001 | .001 | SC | 4 |
| 3 | 0.01 | .01 | SC | 4 |
| 4 | 0.03 | .03 | SC | 4 |
| 5 | 0.1 | 0.1 | SC | 4 |
| 6 | 0.3 | 0.3 | SC | 4 |
| 7 | 1 | 1 | SC | 4 |
| 8 | 3 | 3 | SC | 4 |
| 9 | 0.001 | 0.001 | IV | 4 |
| 10 | 0.03 | 0.03 | IV | 4 |
| 11 | 3 | 3 | IV | 4 |
| 12 | 0.03 | 0.03 | IV | 4 |
| (Positive | | | | |
| Control **) | | | | |

* Negative control (Group 1) animals only received the vehicle (NaCl).
** Positive control (Group 12) animals received hGHRH(1-44) only.

For pharmacodynamic investigations, blood samples (approximately 1.3 ml) were collected from 2 animals per group per time point (maximum 3 time points/animal) via a jugular venipuncture at the following time points: pre-dose, 4, 10, 15, 45 minutes and 5 hours post dosing. All blood samples were collected into potassium EDTA tubes and centrifuged under refrigeration (2 to 8° C., 1500 g for 10 minutes).

ii—Rat Growth Hormone Determination

Plasma GH was determined by Linco Diagnostic Services using their own kit.

Linco's Rat Growth Hormone radioimmunoassay kit (RIA) (RGH-45HK) is intended for the quantitative determination of Rat Growth Hormone in serum, plasma, and tissue culture media. It is a completely homologous assay since the antibody was raised against recombinant Rat Growth Hormone and both the tracer and the standard are prepared with the same recombinant Rat Growth Hormone. The kit includes standards, antibody, tracer, quality controls, precipitating reagents and buffer necessary to complete a RIA. The assay was conducted under the following conditions: overnight; equilibrium incubation at room temperature; sample volume: 100 μl serum, plasma, or cell culture media. The label used was ¹²⁵I-Rat Growth Hormone (20,000 CPM/tube).

The performance of the assay was:

ED₈₀=1.0±0.1 ng/ml

ED₅₀=4.7±0.2 ng/ml

ED₂₀=23.1±0.7 ng/ml

Finally, the specificity of the assay was the following:

Rat Growth Hormone 100%;

Rat Prolactin <0.1%;

Porcine Growth Hormone <0.5%;

Human Growth Hormone <0.1%.

iii—In Vivo Administration into a Male Beagle Dog

Human GHRH analogue # 5, in 0.9% sodium chloride for injection USP, was administered on days 3, 5 and 8 at dose levels of 0.01, 0.1, and 1 mg/kg body weight, respectively by subcutaneous (SC) injection to an approximately 8-month old male dog as shown in Table 3. On Day 1, the dog received the control (vehicle) article and on Day 11, the animal received the positive control, hGHRH (1-44)NH₂ at a dose level of 0.01 mg/kg. Prior to administration, all dosing formulations were filtered using a 0.22 μm filter to ensure sterility. The actual amount of GHRH analogue # 5 administered was calculated and adjusted based on the animal's most recent body weight. Dosing started at approximately the same time each day, commencing at 9:00 am±30 minutes.

TABLE 3
In vivo administration of GHRH analogue # 5 to a male Beagle dog.

| | | | Route of | |
|---|---|---|---|---|
| Dose Level | Dose Conc. | Admin- | Animal | |
| Day | (mg/kg) | (mg/ml) | istration | Number |
| 1 (Negative | 0 | 0 | SC | 1002A |
| Control *) | | | | |
| 3 | 0.01 | 0.01 | SC | 1002A |
| 5 | 0.1 | 0.1 | SC | 1002A |
| 8 | 1.00 | 1.00 | SC | 1002A |
| 11 (Positive | 0.01 | 0.01 | SC | 1002A |
| Control **) | | | | |

* Negative control: the animal received only the vehicle (NaCl).
** Positive control (Day 11): the animal received hGHRH(1-44) only.

For pharmacodynamic investigations, blood samples (approximately 1.0 ml) were collected from the dog on each treatment day via a jugular venipuncture at the following time points: pre-dose, 7, 15, 22, 30, 45, and 60 minutes post dosing. All blood samples were collected into potassium EDTA tubes and centrifuged under refrigeration (2 to 8° C., 1500 g for 10 minutes).

iv—Canine Growth Hormone Determination

Plasma GH was determined by Linco Diagnostic Services using their own kit. Linco's Porcine/Canine Growth Hormone radioimmunoassay kit (RIA) (PGH-46HK) has been developed to quantitate Growth Hormone in plasma, serum, and tissue culture media. It is a completely homologous assay since the antibody was raised against recombinant Porcine Growth Hormone and both the standard and tracer are prepared with recombinant Porcine Growth Hormone. Since the amino acid sequences of Porcine Growth Hormone and Canine Growth Hormone are identical, this assay developed for Porcine Growth Hormone measures Canine Growth Hormone levels with equal efficiency. All components are included (standards, antibody, tracer, quality controls, precipitating reagents and buffer) necessary to complete a RIA. The assay was conducted under the following conditions: overnight; equilibrium incubation at room temperature; sample volume: 100 μl serum, plasma, or cell culture media. The label used was ¹²⁵I-Porcine/Canine Growth Hormone (18,000 CPM/tube).

The performance of the assay was:

ED₈₀=2.3±0.2 ng/ml

ED₅₀=9.8±0.5 ng/ml

ED₂₀=41.8±1.4 ng/ml

Finally, the specificity of the assay was the following:

Porcine Growth Hormone 100%;

Porcine Prolactin <0.1%;

Canine Growth Hormone 100%;

Human Growth Hormone <0.5%.

### Example 3

### In Vitro Proteolytic Resistance of Analogues Compared to hGHRH(1-29)NH₂ in Rat Serum

As presented in Table 4, after a 60-minute incubation period, all GHRH analogues presented significantly higher residual concentrations in comparison with hGHRH(1-29)NH₂. Moreover, the residual concentration of GHRH analogue # 5 was significantly higher than that of either GHRH analogue 1, 2 or 3. Therefore, with the exception of GHRH analogue # 4, these results indicate that GHRH analogue # 5 exhibited the best in vitro resistance to proteolysis, using the described assay.

TABLE 4
In vitro proteolytic resistance of analogues compared
to hGHRH(1-29)NH₂ in rat serum.

| | | Residual |
|---|---|---|
| | Duration of | concentration |
| | incubation | (% of initial |
| Compound | (min) | concentration) |
| Human GHRH(1-29) | 0 | 100 ± 0 |
| NH₂ | 8 | 81 ± 2 |
| (n = 19) | 15 | 66 ± 3 |
| | 30 | 43 ± 2 |
| | 60 | 16 ± 1 |
| GHRH analogue # 1 | 0 | 100 ± 0 |
| (n = 3) | 8 | 75 ± 12 |
| | 15 | 70 ± 15 |
| | 30 | 53 ± 8 |
| | 60 | 30 ± 6 |
| GHRH analogue # 2 | 0 | 100 ± 0 |
| (n = 4) | 8 | 83 ± 3 |
| | 15 | 73 ± 5 |
| | 30 | 53 ± 3 |
| | 60 | 29 ± 2 |
| GHRH analogue # 3 | 0 | 100 ± 0 |
| (n = 4) | 8 | 82 ± 7 |
| | 15 | 88 ± 7 |
| | 30 | 70 ± 12 |
| | 60 | 36 ± 4 |
| GHRH analogue # 4 | 0 | 100 ± 0 |
| (n = 4) | 8 | 98 ± 2 |
| | 15 | 100 ± 0 |
| | 30 | 99 ± 1 |
| | 60 | 97 ± 3 |
| GHRH analogue # 5 | 0 | 100 ± 0 |
| (n = 4) | 8 | 92 ± 5 |
| | 15 | 82 ± 6 |
| | 30 | 74 ± 7 |
| | 60 | 50 ± 3 |

Values represent the mean±SEM of 3 to 4 experiments for the GHRH analogues and the mean±SEM of 19 experiments for hGHRH(1-29)NH₂.

### Example 4

### In Vitro Proteolytic Resistance of Analogues Compared to hGHRH(1-29)NH₂ in Human Plasma and Serum

Referring now to Tables 5 and 6, one can see values of the in vitro proteolytic resistance of hGHRH(1-44)NH₂, hGHRH(1-29)NH₂ and of three GHRH analogues. This resistance is expressed as the mean residual amount of each peptide (in percentage) upon incubation times varying from 0 to 420 minutes in human plasma (Table 5) and human serum (Table 6). More specifically, the values represent the mean, standard deviation and standard error from the mean of 3 to 7 experiments.

As can be particularly appreciated in Table 5, with regard to the native form of GHRH, incubation times varying from 180 to 420-minute led to a significant decrease in the mean residual amount of said peptides. In contrast, after a 180-minute incubation, all three (3) analogues still presented relatively high mean residual amounts (68 to 81%). Moreover, even after a 420-minute incubation, GHRH analogue # 5 still presented 75% of mean residual amount. Using the two-tailed unpaired Student's t test with Welch's correction, with a statistical significance established at P<0.05, a significant difference was observed between the residual amount of analogues compared to human GHRH(1-29)NH₂. Upon further statistical analysis, it was also observed that the residual amount of hGHRH(1-29)NH₂ was significantly lower in human plasma than that of anyone of GHRH analogues # 1, 3 and 5 (P<0.01). However, the mean residual amount of these analogues was not significantly different from one another.

Referring now to Table 6, one can appreciate that upon a 420-minute incubation, while hGHRH(1-29)NH₂ disappeared totally, GHRH analogue # 5 remained at 50% of its initial concentration.

Therefore, upon incubation in both human plasma and human serum, the residual amount of the native form of GHRH was significantly lower than that of its analogues.

TABLE 5
In vitro proteolytic resistance of native
GHRH and GHRH analogues, upon
incubation in human plasma.

| | | Mean | | | |
|---|---|---|---|---|---|
| | IT | residual | | | |
| Peptide | (min) | amount (%) | SD | SEM | n |
| hGHRH (1-44) NH2 | 0 | 100 | 0 | 0 | 3 |
| | 180 | 31 | 1 | 1 | 3 |
| | 420 | 3 | 5 | 3 | 3 |
| hGHRH (1-29) NH2 | 0 | 100 | 0 | 0 | 5 |
| | 60 | 53 | 7 | 4 | 4 |
| | 120 | 44 | 5 | 3 | 4 |
| | 180 | 23 | 15 | 5 | 8 |
| | 420 | 5 | 9 | 5 | 3 |
| (D-Ala-2, Ala-8, Ala-15, Lys-22) | 0 | 100 | 0 | 0 | 4 |
| hGHRH (1-29) NH₂ | 60 | 79 | 7 | 4 | 4 |
| | 120 | 63 | 7 | 4 | 4 |
| | 180 | 68 | 1 | 1 | 3 |
| (D-Ala-2, D-Tyr-10, Lys-22) | 0 | 100 | 0 | 0 | 4 |
| hGHRH (1-29) NH₂ | 60 | 87 | 10 | 5 | 4 |
| | 120 | 78 | 15 | 8 | 4 |
| | 180 | 81 | 11 | 6 | 4 |
| (D-Ala-2, D-Tyr-10, D-Ala-15, | 0 | 100 | 0 | 0 | 4 |
| Lys-22) hGHRH (1-29) NH₂ | 60 | 92 | 10 | 5 | 4 |
| | 120 | 84 | 12 | 6 | 4 |
| | 180 | 78 | 11 | 4 | 7 |
| | 420 | 75 | 3 | 2 | 3 |

IT: incubation time; SEM: standard error from the mean; SD: standard deviation; n: number of experiments.

TABLE 6
In vitro proteolytic resistance
of native GHRH and GHRH analogues,
upon incubation in human serum.

| | | Mean | | | |
|---|---|---|---|---|---|
| | IT | residual | | | |
| Peptide | (min) | amount (%) | SD | SEM | n |
| hGHRH (1-29) NH₂ | 0 | 100 | 0 | 0 | 3 |
| | 60 | 57 | 11 | 6 | 3 |
| | 120 | 37 | 2 | 1 | 3 |
| | 180 | 16 | 10 | 4 | 6 |
| | 420 | 0 | 0 | 0 | 3 |
| (D-Ala-2, D-Tyr-10, D-Ala-15, | 0 | 100 | 0 | 0 | 3 |
| Lys-22) hGHRH (1-29) NH₂ | 60 | 88 | 20 | 12 | 3 |
| | 120 | 76 | 8 | 5 | 3 |
| | 180 | 63 | 5 | 2 | 6 |
| | 420 | 50 | 7 | 4 | 3 |

IT: incubation time; SEM: standard error from the mean; SD: standard deviation; n: number of experiments.

### Example 5

### Binding Affinity of GHRH in its Native and Analogue Forms, to the hGHRH Receptor

As shown in Table 7, no significant difference was observed (two-tailed unpaired Student's t test with Welch's correction, statistical significance established at P<0.05) between the IC₅₀ of human GHRH(1-44)NH₂ and that of GHRH analogue # 5 indicating that this GHRH analogue has an affinity at least as high as the native human GHRH(1-44)NH₂ for the human GHRH receptor.

Values represent the mean±SEM of 3 experiments performed in triplicate for the analogues and the mean±SEM of 2 experiments performed in triplicate for hGHRH(1-44)NH₂. IC₅₀ is the concentration of peptide inhibiting 50% of ¹²⁵I-GHRH specific binding as determined by the LIGAND program for analysis of competition curves.

TABLE 7
In vitro binding affinity of human GHRH
analogue # 5 and hGHRH(1-44)NH₂ in
BHK cell membrane preparations expressing
the human GHRH receptor.

| | | IC₅₀ |
|---|---|---|
| No | Name of compound | (pM) |
| | Human GHRH(1-44)NH₂ | 5.2 ± 3.4 |
| 5 | [D-Ala², D-Tyr¹⁰, D-Ala¹⁵, Lys²²] | 1.2 ± 0.4 |
| | human GHRH(1-29)NH₂ | |

### Example 6

### In Vitro Binding Affinity of hGHRH (1-29)-NH₂ Analogues and hGHRH (1-29)-NH₂ in BHK Cell Membrane Preparations Expressing the Human GHRH Receptor and In Vitro Proteolytic Resistance of the Analogues

For the binding assay results presented in Tables 8 to 11, values represent the mean SEM of 8 independent experiments performed in triplicate for the analogues and the mean±SEM of 4 experiments performed in triplicate for hGHRH(1-29)NH₂. IC₅₀ is the concentration of peptide inhibiting 50% of ¹²⁵I-GHRH specific binding as determined by the LIGAND program for analysis of competition curves. The relative affinity was obtained by taking the ratio IC₅₀ of hGHRH (1-29)-NH₂/IC₅₀ analogue.

For the proteolysis assay results presented in Tables 9 to 11, values represent the mean±SEM of 3 to 5 independent experiments.

As shown in following Table 8, GHRH analogues # 1, 2, 3 and 5 exhibit a significantly higher binding affinity than that of hGHRH(1-29)NH₂ for its receptor. Moreover, although the relative binding affinity of GHRH analogues # 1 and # 5 for the human GHRH receptor do not differ significantly from one another, the affinity of GHRH analogue # 5 is significantly higher than that of # 3.

TABLE 8
In vitro relative binding affinity of
GHRH analogues in BHK cells
expressing the human GHRH receptor.

| | | IC₅₀ | | Relative binding affinity (R1) of |
|---|---|---|---|---|
| | | (molar | | compounds in comparison with |
| | | concen- | | hGHRH(1-29)NH₂in BHK cells |
| No | Name of compound | tration) | | expressing the hGHRH receptor |
| 1 | [D-Ala², Ala⁸, Ala¹⁵, | 33 ± 12 | pM | 499 ± 234 |
| | Lys²²]human GHRH | | | |
| | (1-29)NH₂ | | | |
| 2 | [Ala⁸, Ala⁹ Ala¹⁵, | 0.77 ± 0.09 | nM | 3.70 ± 0.52 |
| | Ala²²,]human GHRH | | | |
| | (1-29)NH₂ | | | |
| 3 | [D-Ala², D-Tyr¹⁰, | 6.3 ± 1.1 | pM | 239 ± 55 |
| | Lys²²]human GHRH | | | |
| | (1-29)NH₂ | | | |
| 4 | [D-Ala², Ala⁸, D-Tyr¹⁰, | 37 ± 4 | nM | 0.05 ± 0.01 |
| | Ala¹⁵, D-Lys²¹, Lys²²] | | | |
| | human GHRH(1-29)NH₂ | | | |
| 5 | [D-Ala², D-Tyr¹⁰, | 6.0 ± 2.4 | pM | 939 ± 249 |
| | D-Ala¹⁵, Lys²²] | | | |
| | human GHRH(1-29)NH₂ | | | |

TABLE 9
In vitro potency index of GHRH analogues
after 60-min incubation in human plasma.

| | | | | | in |
|---|---|---|---|---|---|
| | | | | | vitro |
| | | Resid- | | | po- |
| | | ual | | | tency |
| | | peptide | | | index |
| | | concen- | | | (R1 × |
| No | Name of compound | tration* | R1 | R2 | R2) |
| 1 | [D-Ala², Ala⁸, Ala¹⁵, | 79 ± 4 | 499 ± 234 | 1.52 ± 0.18 | 758 |
| | Lys²²]human GHRH | | | | |
| | (1-29)NH₂ | | | | |
| 2 | [Ala⁸, Ala⁹Ala¹⁵, | Not | 3.70 ± 0.52 | Not | Not |
| | Ala²²]human | tested | | tested | tested |
| | GHRH(1-29)NH₂ | | | | |
| 3 | [D-Ala², D-Tyr¹⁰, | 87 ± 5 | 239 ± 55 | 1.69 ± 0.22 | 404 |
| | Lys²²]human GHRH | | | | |
| | (1-29)NH₂ | | | | |
| 4 | [D-Ala², Ala⁸, | Not | 0.05 ± 0.01 | Not | Not |
| | D-Tyr¹⁰, | tested | | tested | tested |
| | Ala¹⁵, D-Lys²¹, Lys²²] | | | | |
| | human GHRH | | | | |
| | (1-29)NH₂ | | | | |
| 5 | [D-Ala², D-Tyr¹⁰, D- | 92 ± 5 | 939 ± 249 | 1.78 ± 0.22 | 1671 |
| | Ala¹⁵, Lys²²] human | | | | |
| | GHRH(1-29)NH₂ | | | | |

*% of initial content at time 0; R1: Relative binding affinity of compounds in comparison with hGHRH(1-29)NH₂in BHK cells expressing the hGHRH receptor; R2: Relative resistance to in vitro proteolysis of compounds in comparison with hGHRH(1-29)NH₂

As can be seen in Table 9, the in vitro potency index of GHRH analogues # 1, 3 and 5 reaches values of 758, 404 and 1671, respectively. In other words, these three (3) analogues have simultaneously a significantly higher binding affinity to their receptor as well as a significantly better resistance to proteolysis upon an in vitro 60-min incubation in human plasma, in comparison with the native hGHRH(1-29)NH2. Moreover, as can be seen in Table 10 below, the in vitro potency index of GHRH analogues is even higher upon a 180-min incubation in human plasma.

TABLE 10
In vitro potency index of GHRH analogues
after 180-min incubation in human plasma.

| | | | | | in |
|---|---|---|---|---|---|
| | | | | | vitro |
| | | Resid- | | | po- |
| | | ual | | | tency |
| | | peptide | | | index |
| | | concen- | | | (R1 × |
| No | Name of compound | tration* | R1 | R2 | R2) |
| 1 | [D-Ala², Ala⁸, Ala¹⁵, | 68 ± 1 | 499 ± 234 | 2.96 ± 0.02 | 1477 |
| | Lys²²]human GHRH | | | | |
| | (1-29)NH₂ | | | | |
| 2 | [Ala⁸, Ala⁹Ala¹⁵, | Not | 3.70 ± 0.52 | Not | Not |
| | Ala²²]human | tested | | tested | tested |
| | GHRH(1-29)NH₂ | | | | |
| | [D-Ala², D-Tyr¹⁰, | 81 ± 1 | 239 ± 55 | 3.54 ± 0.23 | 846 |
| | Lys²²]human GHRH | | | | |
| | (1-29)NH₂ | | | | |
| 4 | [D-Ala², Ala⁸, | Not | 0.05 ± 0.01 | Not | Not |
| | D-Tyr¹⁰, | tested | | tested | tested |
| | Ala¹⁵, D-Lys²¹, Lys²²] | | | | |
| | human GHRH | | | | |
| | (1-29)NH₂ | | | | |
| 5 | [D-Ala², D-Tyr¹⁰, D- | 74 ± 7 | 939 ± 249 | 3.21 ± 0.31 | 3014 |
| | Ala¹⁵, Lys²²] human | | | | |
| | GHRH(1-29)NH₂ | | | | |

*% of initial content at time 0; R1: Relative binding affinity of compounds in comparison with hGHRH(1-29)NH₂ in BHK cells expressing the hGHRH receptor ± SEM; R2: Relative resistance to in vitro proteolysis of compounds in comparison with hGHRH(1-29)NH₂ ± SEM.

The next step was to test whether the same observations held true after incubation in human serum. Results for GHRH analogue # 5 can be seen in Table 11. Again, upon 60 or 180 minutes of incubation in human serum, the GHRH analogue # 5 still presented a significantly higher in vitro potency index, compared to the native hGHRH(1-29)NH₂.

TABLE 11
In vitro potency index of GHRH analogue
# 5 after 60 and 180-min incubation
in human serum.

| | | in vitro | Resid- | | in |
|---|---|---|---|---|---|
| | | potency | ual | | vitro |
| | | index | peptide | | po- |
| | | (R1 × | concen- | | tency |
| | | R2) | tration | | index |
| | R2 | (60 | (180 | R2 | (R1 × R2) |
| R1 | (60 min) | min) | min)* | (180 min) | (180 min) |
| 939 ± 249 | 1.55 ± 0.04 | 1455 | 62 ± 2 | 2.93 ± 0.87 | 2751 |

*% of initial content at time 0: R1: Relative binding affinity of compounds in comparison with hGHRH(1-29)NH₂in BHK cells expressing the hGHRH receptor ± SEM; R2: Relative resistance to in vitro proteolysis of compounds in comparison with hGHRH(1-29)NH₂ ± SEM.

### Example 7

### Use of the GHRH Analogue for the Specific Stimulation of In Vivo GH Release

The present invention is directed to the use of the GHRH analogue for the specific stimulation of in vivo GH release. Such a use is based upon the following background.

Integration of all the factors that affect GH synthesis and secretion lead to a pulsatile pattern of release, thus a single measurement of plasma GH levels is difficult to interpret. Basal concentrations of GH in blood are very low. In children and young adults, the most intense period of growth hormone release is shortly after the onset of deep sleep. The pattern of GH secretion is episodic, with six to eight pulses per day and very low levels between pulses and is linked to stages 3 and 4 of the sleep cycle, but this association is less evident with increasing age. Some of these pulses are associated with meals, stress, exercise, or slow-wave sleep.

GH pulses occur more frequently and the basal level of plasma GH is higher in females than males who have fewer GH pulses but which are of higher amplitude. In humans there is typically one high secretion pulse and a few lower ones during the 24-h day-night span. Delay, advance or interruption of a sleep phase will shift the main GH secretion pulse correspondingly. At least in humans, GH secretion is also controlled by an endogenous circadian rhythm. When the sleep period is shifted from its normal time, some GH is still secreted during the early night according to the endogenous clock. GH secretion is highest during growing and early adulthood. In humans, the secretion rate starts to decrease during the fourth decade of life. During aging the daytime secretion pulses diminish first, while the sleep-associated GH pulse persists.

In animals, it is more difficult to find a correlation between GH secretion and sleep because many animal species have typically several sleep phases of variable lengths during the 24-h day-night span. However, elevated plasma GH levels during sleep have been demonstrated in several mammals (reviewed by Van Cauter, E. et al. Sleep, 1998, 21: 553-566). In the rat, which is a widely used animal model in neuroscience, the GH secretion is pulsatile with an approximately 3.3-h cycle. This rhythm is associated with an ultradian sleep-wake rhythm with the same cycle length, so that the GH pulses precede the sleep maxima by about 24 min (Mitsugi, N. and Kimura, F. NeuroendocrinoL, 1985, 41: 125-130). Short-term (3 h) total sleep deprivation during the light phase resulted in a decrease of GH secretion during the deprivation in the rat (Kimura, F. and Tsai, C.-W. J. Physiol. (Lond.), 1984, 353: 305-315).

In order to assess such use of the GHRH analogues, the following experiments were undertaken. More specially, the goal was to assess the pharmacodynamic and pharmacokinetic profiles and acute toxicity of GHRH analogue # 5 when administered once by subcutaneous or intravenous injection to female Sprague-Dawley rats followed by a 14-day observation period and the pharmacodynamic profile in a male Beagle dog when the GHRH analogue was administered at escalating doses to the same dog by subcutaneous injection with at least 2-day washout period. The above GHRH analogue is a variation of a synthetic acetate salt of an amidated synthetic 29-amino acid peptide that corresponds to the amino-terminal segment of the naturally-occurring human growth hormone—releasing hormone (GHRH) with four amino acid substitutions in positions 2, 10, 15, and 22.

Experimental Results

i. Rat Study

Each sample was blind tested in duplicate and the result represents the mathematical mean of two. The source of plasma and samples was unknown to the analyst.

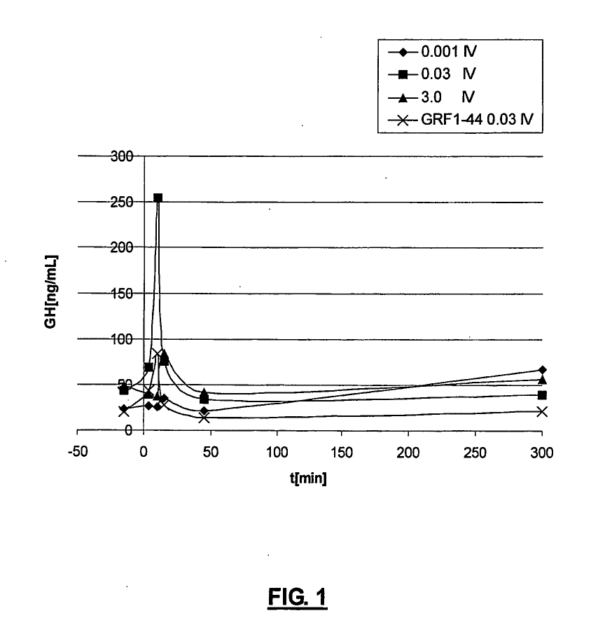

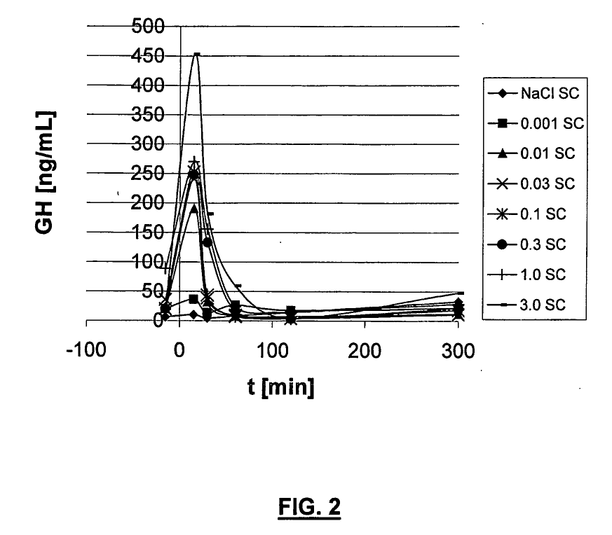

The results of rat plasma testing for rat GH are presented in Table 12 below. Each value in the Table 12 represents the mathematical mean of two animals. The same data were then plotted against time and pharmacodynamic curves are presented in FIG. 1 for the intravenous and in FIG. 2 for the subcutaneous administrations.

Growth hormone areas under the curves (AUC) for different time duration are presented in Table 13.

The data show that both intravenous and subcutaneous administrations of GHRH analogue # 5 elicited a dose-dependent response: secretion of GH into peripheral blood. Significant inter-animal variation in GH level was observed. This confirms the observations of others.

Most of the animals exhibited elevated pre-administration concentration of circulating growth hormone. There was a trend for GH concentration to go up again at about 300 minutes (5 hours) post GHRH or NaCl injection in all groups of rats.

TABLE 12
Amplitude of Rat Growth Hormone Secretion, at various time points,
in response to GHRH analogue # 5 administration in adult female rats.

| GHRH | | Plasma Rat Growth Hormone (ng/ml) | | | | | | | | |
|---|---|---|---|---|---|---|---|---|---|---|
| mg/kg | | Time post-GHRH administration (minutes) | | | | | | | | |
| BW Route | | −120 | 4 | 10 | 15 | 30 | 45 | 60 | 120 | 300 |
| NaCl | SC | 6.55 | ND | ND | 10.15 | 4.85 | ND | 8.55 | 14.65 | 32.79 |
| 0.001 | SC | 20.15 | ND | ND | 36.7 | 14.2 | ND | 26.85 | 17.8 | 21.85 |
| 0.01 | SC | 20.4 | ND | ND | 190.4 | 31.9 | ND | 8.5 | 7.6 | 11.95 |
| 0.03 | SC | 36.1 | ND | ND | 240.9 | 39.05 | ND | 9.5 | 4.6 | 11.25 |
| 0.1 | SC | 20.4 | ND | ND | 252.4 | 43.8 | ND | 7.65 | 4.45 | 18.7 |
| 0.3 | SC | 20.7 | ND | ND | 247.9 | 133.5 | ND | 16.75 | 4.00 | 21.8 |
| 1.00 | SC | 88.95 | ND | ND | 270.0 | 155.85 | ND | 24.35 | 15.85 | 28.85 |
| 3.00 | SC | 20.05 | ND | ND | 453.0 | 181.55 | ND | 59.4 | 4.45 | 47.9 |
| 0.001 | IV | 23.85 | 26.2 | 25.85 | 34.65 | ND | 21.15 | ND | ND | 67.15 |
| 0.03 | IV | 43.15 | 68.45 | 254.65 | 75.1 | ND | 33.4 | ND | ND | 38.75 |
| 3.0 | IV | 48.6 | 38.7 | 36.95 | 83.65 | ND | 41.6 | ND | ND | 56.7 |

| | | | | | | | | | |
|---|---|---|---|---|---|---|---|---|---|
| GHRH | 20.2 | 43.7 | 83.9 | 27.9 | ND | 14.1 | ND | ND | 21.7 |
| (1-44) | | | | | | | | | |

| | |
|---|---|
| 0.03 | IV |

BW: body weight; ND: not determined.

As shown in Table 12, Rat Growth Hormone (ng/mL) was measured in duplicate. Values represent the mean of two animals per time point. The Route represents the route of administration which was either subcutaneous (SC) or intravenous (IV).

TABLE 13
Cumulative Rat Growth Hormone Secretion in adult female
rats in response to GHRH analogue # 5 administration,
as determined by GH Area Under the Curve (AUC).

| GHRH mg/kg | | GH AUC | | GHRH mg/kg | | GH AUC | |
|---|---|---|---|---|---|---|---|
| BW SC Route | | 120 min | 300 min | BW IV Route | | 45 min | 300 min |
| NaCl | SC | 1165 | 5434 | 0.001 | IV | 1197 | 12280 |
| 0.001 | SC | 2914 | 6482 | 0.03 | IV | 3407 | 12060 |
| 0.01 | SC | 5153 | 6913 | 3.0 | IV | 2384 | 14299 |
| 0.03 | SC | 6192 | 7618 | GHRH | | 1380 | 5754 |
| | | | | (1-44) | | | |
| | | | | 0.03 | IV | | |
| 0.1 | SC | 6423 | 8507 | | | | |
| 0.3 | SC | 8636 | 10958 | | | | |
| 1.0 | SC | 10425 | 14448 | | | | |
| 3.0 | SC | 15562 | 20273 | | | | |

BW: body weight.

As shown in Table 13, the Route represents the route of administration which is either subcutaneous (SC) or intravenous (IV). Furthermore, GH AUC was determined 45, 120 or 300 minutes post-GHRH administration.

i. Dog Study

Each sample was blind tested in duplicate and the result represents the mathematical mean of two. The source of plasma and samples was unknown to the analyst.

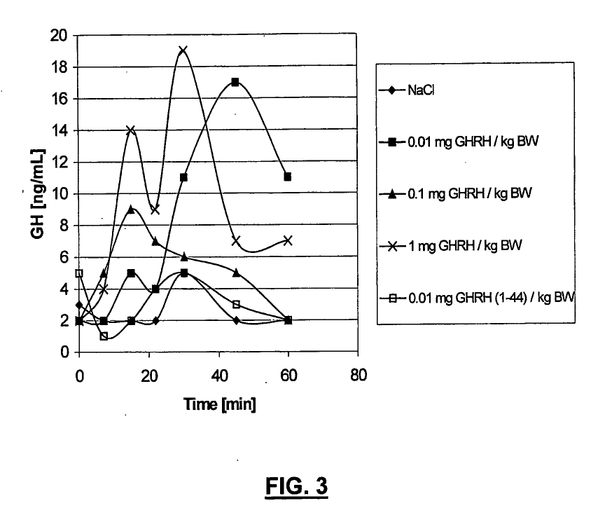

The results of canine plasma testing for canine GH are presented in Table 14 below. The same data were then plotted against time and pharmacodynamic curves are presented in FIG. 3 for the subcutaneous administrations.

The data show that subcutaneous administrations of GHRH analogue # 5 elicited a dose-dependent response: secretion of GH into peripheral blood.

There was a trend for GH concentration to go up again at about 30 or 50 minutes post GHRH administration depending on the dose injected.

No treatment-related clinical signs were observed following GHRH analogue administration into both rats and the dog.

TABLE 14
Amplitude of Canine Growth Hormone Secretion,
at various time points, in an 8-month-old
Beagle dog in response to
GHRH analogue # 5 administration.

| GHRH | | Canine Growth Hormone (ng/ml) | | | | | | |
|---|---|---|---|---|---|---|---|---|
| mg/kg | | Time post-GHRH administration (minutes) | | | | | | |
| BW Route | | 0 | 7 | 15 | 22 | 30 | 45 | 60 |
| NaCl | SC | 3 | 1.99 | 1.99 | 1.99 | 5 | 1.99 | 1.99 |
| 0.01 | SC | 1.99 | 1.99 | 5 | 4 | 11 | 17 | 11 |
| 0.1 | SC | 1.99 | 5 | 9 | 7 | 6 | 5 | 1.99 |
| 1 | SC | 1.99 | 4 | 14 | 9 | 19 | 7 | 7 |

| | | | | | | | |
|---|---|---|---|---|---|---|---|
| hGHRH | 5 | 1 | 1.99 | 4 | 5 | 3 | 1.99 |
| (1-44) | | | | | | | |

| | |
|---|---|
| 0.01 | SC |

Data Interpretation

The data presented above clearly demonstrate that the synthetic GHRH analogue # 5 recognizes GHRH receptors in both rat and dog pituitary and triggers GH response and secretion into circulation. In a rat, the response is dose-dependent both in terms of height of peak amplitude and AUC for the peak duration. The peak secretion following single subcutaneous injection is between 10-15 minutes and 4-10 minutes following intravenous injection. GH secretion in response to GHRH analogue # 5 is twice larger than GH secretion in response to natural hGHRH(1-44)NH₂ both in terms of pulse amplitude and AUC. The highest GHRH analogue # 5 single IV dose induced transient somatotroph desensitization.

In the dog, like in the rat, GH secretion in response to GHRH analogue # 5 is dose-dependent. The peak secretion following single subcutaneous injection is between 5 and 15 minutes and there clearly is a second GH peak not observed in response to saline or native GHRH indicating longer stability of the analogue in canine plasma. GH response to GHRH analogue # 5 is significantly larger than GH secretion in response to natural hGHRH(1-44)NH₂ (AUC not measured).

### CONCLUSIONS

In vivo proof-of-concept has been established. GHRH(1-29)NH₂ synthetic analogue of the amino acid sequence of H-Tyr D-Ala2 Asp Ala Ile Phe Thr Asn Ser D-Tyr10 Arg Lys Val Leu D-Ala15 Gin Leu Ser Ala Arg Lys Lys22 Leu Gin Asp Ile Met Ser Arg-NH₂ in which Ala2, Tyr10, Gly15, and Leu22 have been replaced by D-Ala2, D-Tyr10, D-Ala15, and Lys22 binds to GHRH receptor on somatotrophs in rat and dog pituitaries and stimulates secretion and release of growth hormone in a dose-dependent manner.

GHRH analogue # 5 is at least two times more potent in vivo than the natural 44 amino acid GHRH.

1. A GHRH analogue, a functional derivative of said analogue, or a pharmaceutically acceptable salt thereof comprising formula X: Tyr-A2-Asp-Ala-Ile-Phe-Thr-A8-A9-A 10-Arg-Lys-Val-Leu-A 15-Gln-Leu-Ser-Ala-Arg-A21-A22-Leu-Gln-Asp Ile-Met-Ser-Arg-A30-NH₂, wherein
A2 is Ala or D-Ala;
A8 is Asn, D-Asn or Ala;
A9 is Ser or Ala;
A10 is Tyr or D-Tyr;
A15 is Gly, Ala or D-Ala;
A21 is Lys or D-Lys;
A22 is Leu, D-Leu, Lys or Ala; and
A30 is a bond or any amino acid sequence of 1 up to 15 residues;
said analogue, functional derivative of said analogue or salt thereof having an in vitro potency index substantially higher than the in vitro potency index of a naturally occurring GHRH.

2.-16. (canceled)

17. A GHRH analogue or a pharmaceutically acceptable salt thereof able to stimulate secretion or synthesis of growth hormone in a mammal, said GHRH analog or pharmaceutically acceptable salt having an in vitro potency index substantially higher than the in vitro potency index of a native hGHRH1-29 and having formula Tyr-D-Ala²-Asp-Ala-Ile-Phe-Thr-Asn-Ser-D-Tyr¹⁰-Arg-Lys-Val-Leu-D-Ala¹⁵-Gln-Leu-Ser-Ala-Arg-Lys-Lys²²-Leu-Gln-Asp-Ile-Met-Ser-Arg-A30-NH₂, wherein A30 is a bond or any amino acid sequence of 1 up to 15 residues.

18. A GHRH analogue according to claim 17, wherein the in vitro potency index is at least 500-fold higher than the in vitro potency index of a native hGHRH1-29.

19. The GHRH analogue of claim 18, wherein the in vitro potency index is at least 1500-fold higher than the in vitro potency index of a native hGHRH1-29.

20. The GHRH analogue of claim 19, wherein the in vitro potency index is at least 2500-fold higher than the in vitro potency index of a native hGHRH1-29.

21. The GHRH analogue of claim 17, wherein said GHRH analogue has the formula Tyr-D-Ala²-Asp-Ala-Ile-Phe-Thr-Asn-Ser-D-Tyr¹⁰-Arg-Lys-Val-Leu-D-Ala¹⁵-Gln-Leu-Ser-Ala-Arg-Lys-Lys 22-Leu-Gln-Asp-Ile-Met-Ser-Arg-NH₂.

22. A pharmaceutical composition, comprising:
a) an effective amount of a GHRH analogue or a pharmaceutically acceptable salt thereof comprising formula X: Tyr-A2-Asp-Ala-Ile-Phe-Thr-A8-Ser-A10-Arg-Lys-Val-Leu-A15-Gln-Leu-Ser-Ala-Arg-Lys-A22-Leu-Gln-Asp-Ile-Met-Ser-Arg-A30-NH₂, wherein
A2 is Ala or D-Ala;
A8 is Asn, D-Asn or Ala;
A10 is Tyr or D-Tyr;
A15 is Gly, Ala or D-Ala;
A22 is Leu, D-Leu, Lys or Ala; and
A30 is a bond or any amino acid sequence of 1 up to 15 residues and wherein said analogue comprises at least one amino acid substitution in the native form of hGHRH1-29; and;
b) a pharmaceutically acceptable carrier.

23. The pharmaceutical composition of claim 22, wherein said GHRH analogue or salt thereof is selected from the group consisting of, and wherein:
A2 is D-Ala, A8 is Ala, A15 is Ala, A22 is Lys;
A2 is D-Ala, A10 is D-Tyr, and A22 is Lys and;
A2 is D-Ala, A10 is D-Tyr, A15 is D-Ala, and A22 is Lys.

24. The pharmaceutical composition of claim 23, wherein A2 is D-Ala, A8 is Asn, A10 is D-Tyr, A15 is D-Ala, A22 is Lys and A30 is a bond.

25. A pharmaceutical composition, comprising:
a) an effective amount of a GHRH analogue or a pharmaceutically acceptable salt thereof of formula X: Tyr-A2-Asp-Ala-Ile-Phe-Thr-A8-Ser-A10-Arg-Lys-Val-Leu-A15-Gln-Leu-Ser-Ala-Arg-Lys-A22-Leu-Gln-Asp-Ile-Met-Ser-Arg-A30-NH₂, wherein
A2 is Ala or D-Ala;
A8 is Asn, D-Asn or Ala;
A10 is Tyr or D-Tyr;
A15 is Gly, Ala or D-Ala;
A22 is Leu, D-Leu, Lys or Ala; and
A30 is a bond or any amino acid sequence of 1 up to 15 residues and wherein said analogue comprises at least one amino acid substitution in the native form of hGHRH1-29; and;
b) a pharmaceutically acceptable carrier.

26. The pharmaceutical composition of claim 25, wherein said GHRH analogue or salt thereof is selected from the group consisting of, and wherein:
A2 is D-Ala, A8 is Ala, A15 is Ala, A22 is Lys;
A2 is D-Ala, A10 is D-Tyr, and A22 is Lys and;
A2 is D-Ala, A10 is D-Tyr, A15 is D-Ala, and A22 is Lys.

27. The pharmaceutical composition of claim 26, wherein A2 is D-Ala, A8 is Asn, A10 is D-Tyr, A15 is D-Ala, A22 is Lys and A30 is a bond.

28. A pharmaceutical composition for stimulating secretion or synthesis of growth hormone in a mammal in need thereof, the pharmaceutical composition comprising:
a) an effective amount of a GHRH analogue or a pharmaceutically acceptable salt thereof comprising formula X: Tyr-A2-Asp-Ala-Ile-Phe-Thr-A8-Ser-A10-Arg-Lys-Val-Leu-A15-Gln-Leu-Ser-Ala-Arg-Lys-A22-Leu-Gln-Asp-Ile-Met-Ser-Arg-A30-NH₂, wherein
A2 is Ala or D-Ala;
A8 is Asn, D-Asn or Ala;
A10 is Tyr or D-Tyr;
A15 is Gly, Ala or D-Ala;
A22 is Leu, D-Leu, Lys or Ala; and
A30 is a bond or any amino acid sequence of 1 up to 15 residues and wherein said analogue comprises at least one amino acid substitution in the native form of hGHRH 1-29, and;
b) a pharmaceutically acceptable carrier.

29. The pharmaceutical composition of claim 28, wherein said GHRH analogue or salt thereof is selected from the group consisting of, and wherein:
A2 is D-Ala, A8 is Ala, A15 is Ala, A22 is Lys;
A2 is D-Ala, A10 is D-Tyr, and A22 is Lys and;
A2 is D-Ala, A10 is D-Tyr, A15 is D-Ala, and A22 is Lys.

30. The pharmaceutical composition of claim 29, wherein A2 is D-Ala, A8 is Asn, A10 is D-Tyr, A15 is D-Ala, A22 is Lys and A30 is a bond.

31. The use of a GHRH analogue, or a pharmaceutically acceptable salt thereof in the preparation of a pharmaceutical composition for stimulating secretion or synthesis of growth hormone in a mammal in need thereof, said GHRH analog or pharmaceutically acceptable salt comprising formula X: Tyr-A2-Asp-Ala-Ile-Phe-Thr-A8-Ser-A10-Arg-Lys-Val-Leu-A15-Gln-Leu-Ser-Ala-Arg-Lys-A22-Leu-Gln-Asp-Ile-Met-Ser-Arg-A30-NH₂, wherein
A2 is Ala or D-Ala;
A8 is Asn, D-Asn or Ala;
A10 is Tyr or D-Tyr;
A15 is Gly, Ala or D-Ala;
A22 is Leu, D-Leu, Lys or Ala; and
A30 is a bond or any amino acid sequence of 1 up to 15 residues and wherein said analogue comprises at least one amino acid substitution in the native form of hGHRH 1-29.

32. The use as defined in claim 31, wherein said GHRH analogue or salt thereof is selected from the group consisting of, and wherein:
A2 is D-Ala, A8 is Ala, A15 is Ala, A22 is Lys;
A2 is D-Ala, A10 is D-Tyr, and A22 is Lys and;
A2 is D-Ala, A10 is D-Tyr, A15 is D-Ala, and A22 is Lys.

33. The use as defined in claim 32, wherein A2 is D-Ala, A8 is Asn, A10 is D-Tyr, A15 is D-Ala, A22 is Lys and A30 is a bond.

34. The use according to claim 31, wherein said mammal has a disorder selected from the group consisting of hypothalamic pituitary dwarfism, burns, osteoporosis, renal failure, non-union bone-fracture, acute/chronic debilitating illness or infection, wound healing, reduction of the incidence of post-surgical problems, lactation failure, infertility in women, cachexia in cancer patients, anabolic and/or catabolic problems, T-cell immunodeficiencies, neurodegenerative conditions, GHRH receptor-dependent tumors, aging, sleep disorders, muscle wasting diseases such as-in sarcopenic patients, frail elderlies, HIV patients and cancer patients having radiotherapy and chemotherapy side-effects.

35. The use according to claim 34, wherein said muscle wasting diseases are selected from the group consisting of; sarcopenia, frailty in elderlies, HIV and cancer.

36. The use of a GHRH analogue, or a pharmaceutically acceptable salt thereof for stimulating secretion or synthesis of growth hormone in a mammal in need thereof, said GHRH analog or pharmaceutically acceptable salt comprising formula X: Tyr-A2-Asp-Ala-Ile-Phe-Thr-A8-Ser-A 10-Arg-Lys-Val-Leu-A15-Gln-Leu-Ser-Ala-Arg-Lys-A22-Leu-Gln-Asp-Ile-Met-Ser-Arg-A30-NH₂, wherein
A2 is Ala or D-Ala;
A8 is Asn, D-Asn or Ala;
A10 is Tyr or D-Tyr;
A15 is Gly, Ala or D-Ala;
A22 is Leu, D-Leu, Lys or Ala; and
A30 is a bond or any amino acid sequence of 1 up to 15 residues and wherein said analogue comprises at least one amino acid substitution in the native form of hGHRH1-29.

37. The use according to claim 36, wherein said mammal has a disorder selected from the group consisting of hypothalamic pituitary dwarfism, burns, osteoporosis, renal failure, non-union bone-fracture, acute/chronic debilitating illness or infection, wound healing, reduction of the incidence of post-surgical problems, lactation failure, infertility in women, cachexia in cancer patients, anabolic and/or catabolic problems, T-cell immunodeficiencies, neurodegenerative conditions, GHRH receptor-dependent tumors, aging, sleep disorders, muscle wasting diseases such as-in-sarcopenic patients, frail elderlies, HIV patients and cancer patients having radiotherapy and chemotherapy side-effects.

38. The use according to claim 37, wherein said muscle wasting diseases are selected from the group consisting of; sarcopenia, frailty in elderlies, HIV and cancer.
